(19) United States
(12) Patent Application Publication
(10) Pub. No.: US 2024/0255476 A1
(43) Pub. Date: Aug. 1, 2024

(54) IDENTIFICATION METHOD OF MATURITY OF ACACIA HONEY

(71) Applicant: INSTITUTE OF APICULTURAL RESEARCH, CHINESE ACADEMY OF AGRICULTURAL SCIENCES, Beijing (CN)

(72) Inventors: Wenli TIAN, Beijing (CN); Wenqian WANG, Beijing (CN); Xiaofeng XUE, Beijing (CN); Sha YAN, Beijing (CN); Xuan WANG, Beijing (CN); Wenjun PENG, Beijing (CN); Liming WU, Beijing (CN); Kai WANG, Beijing (CN); Xiaoming FANG, Beijing (CN); Xiangxin LI, Beijing (CN)

(73) Assignee: INSTITUTE OF APICULTURAL RESEARCH, CHINESE ACADEMY OF AGRICULTURAL SCIENCES, Beijing (CN)

(21) Appl. No.: 18/566,501

(22) PCT Filed: Nov. 22, 2022
(86) PCT No.: PCT/CN2022/133567

§ 371 Date: Dec. 1, 2023

(30) Foreign Application Priority Data
Nov. 23, 2021 (CN) 202111390505.X

(51) Int. Cl. G01N 30/74; B01D 15/42; G01N 30/02; G01N 30/86; G01N 33/02

(52) U.S. Cl. CPC G01N 30/74; B01D 15/426; G01N 30/8624; G01N 33/02; G01N 2030/027

(57) ABSTRACT

The present disclosure relates to the technical field of food detection, in particular to an identification method of maturity of acacia honey. In the present disclosure, the identification method of maturity of acacia honey includes the following steps: detecting an acacia honey sample using turanose as a characteristic marker; where if the acacia honey sample to be identified has a turanose content of no less than 0.5%, it is determined that the acacia honey sample to be identified is mature acacia honey; if the acacia honey sample to be identified has a turanose content of no less than 0.3% but less than 0.5%, it is determined that the acacia honey sample to be identified is semi-immature acacia honey; and if the acacia honey sample to be identified has a turanose content of less than 0.3%, it is determined that the acacia honey sample to be identified is immature acacia honey.

### CROSS REFERENCE TO RELATED APPLICATION

The present application is a national stage application of International Patent Application NO. PCT/CN2022/133567, filed on Nov. 22, 2022, which claims priority to the Chinese Patent Application No. CN202111390505.X, filed with the China National Intellectual Property Administration (CNIPA) on Nov. 23, 2021, and entitled “IDENTIFICATION METHOD OF MATURITY OF ACACIA HONEY”, both of which are incorporated by references in their entireties.

### TECHNICAL FIELD

The present disclosure relates to the technical field of food detection, in particular to an identification method of maturity of acacia honey.

### BACKGROUND

Acacia honey is made by bees from the nectar of acacia. Acacia honey has a watery white color, a soft, agreeable sweet taste, and a unique fragrance of acacia flowers. Acacia honey is a kind of high-quality and high-end honey, showing a significantly higher market value than that of other honeys. The ripening of acacia honey begins when the bees have finished collecting the nectar. Foraging bees ingest the nectar of acacia flowers into honey sacs and bring back to the hive, where the brewing of acacia honey begins. During the collection, the foraging bees secrete invertase and amylase to be mixed with the nectar. After returning to the hive, the foraging bees spit out and distribute honeydew to the house bees. The house bees suck the honeydew into their honey sac, and conducting repeatedly suction and spitting. During this repeated process, the house bees continuously add various enzymes to realize composition transformation of the honeydew. The house bees keep vibrating their wings when swallowing, spitting, and transforming, so as to accelerate the evaporation of water. When honey in the honeycomb has moisture content down to about 20%, the bees seal the honey with beeswax. At this time, the acacia honey is basically mature. Normally, it takes at least 7 d for the acacia honey to mature, and the honeycomb sealing reaches not less than 75%. Therefore, acacia honey that has been brewed in the hive for at least 7 d with honeycomb sealing no less than 75% is generally considered to be mature acacia honey.

However, acacia has a short flowering period. In order to maximize the yield, acacia nectar of more than 1 d to 3 d is generally collected manually, and its moisture content is reduced to no more than 21% by artificial concentration. In this way, immature acacia honey is obtained.

The physical and chemical indicators of this kind of immature acacia honey meet the requirements of those in national standard acacia honey. As a result, it is difficult to effectively distinguish immature acacia honey from naturally-mature acacia honey, and it is also difficult to determine whether the acacia honey is mature and to identify its maturity. At present, the value of acacia honey with different levels of maturity varies on the market. In order to pursue high value, some companies sell immature or incomplete mature acacia honey as mature acacia honey, and seriously disrupt the order of honey market. In view of this, there is an urgent need to develop stable and reliable indicators to determine the maturity of acacia honey, so as to differentiate acacia honey with different qualities, thereby ensuring the quality and value of acacia honey.

### SUMMARY

An objective of the present disclosure is to provide an identification method of maturity of acacia honey by using turanose as a characteristic marker.

The material composition of honey changes during maturing, but not all chemical components with a high content in the honey undergo characteristic content changes during the maturing. Many components show content changes that have no rules to follow, and are difficult to be characteristic markers of mature honey. Honey adulteration and honey maturity are two different evaluation methods of honey. Compared with adulteration, the evaluation of maturity can achieve product classification very well. That is, in the case of not involving the adulteration, products are divided into different grades according to their maturity, thus achieving different values of the products. The identification and evaluation of maturity plays an important supporting role in realizing the high quality and good price of honey. In the present disclosure, researches on the maturing of acacia honey have found that the content change of turanose during the maturing of acacia honey has obvious correlation with the brewing time and sealing in the hive. Therefore, the turanose can be used as a characteristic marker to identify mature and immature acacia honey. Furthermore, through the detection of large samples for acacia honey with different years, different sources, and different maturity levels, it is found that the turanose shows extremely-stable content changing characteristics in acacia honey with different maturity levels. Meanwhile, due to a high cost, the turanose is difficult to be artificially added to acacia honey. This also indicates that turanose is suitable as a characteristic marker of the maturity of acacia honey for identifying mature and immature acacia honey.

Based on the above findings, the present disclosure provides the following technical solutions:

In a first aspect, the present disclosure provides use of turanose as a characteristic marker of mature acacia honey.

In a second aspect, the present disclosure provides use of turanose in identifying maturity of acacia honey.

In a third aspect, the present disclosure provides use of turanose in identifying mature acacia honey and immature acacia honey.

In a fourth aspect, the present disclosure provides an identification method of maturity of acacia honey, including the following steps: detecting an acacia honey sample to be identified using turanose as a characteristic marker; where if the acacia honey sample to be identified has a turanose content of no less than 0.5%, it is determined that the acacia honey sample to be identified is mature acacia honey; if the acacia honey sample to be identified has a turanose content of no less than 0.3% but less than 0.5%, it is determined that the acacia honey sample to be identified is semi-mature acacia honey; and if the acacia honey sample to be identified has a turanose content of less than 0.3%, it is determined that the acacia honey sample to be identified is immature acacia honey;
the mature acacia honey is brewed in a hive for at least 7 d, and shows honeycomb sealing of no less than 75%;
the semi-immature acacia honey is brewed in the hive for more than 3 d but no less than 5 d, and shows honeycomb sealing of 30% to 70%; and
the immature acacia honey is brewed in the hive for 0 d to 3 d, and shows honeycomb sealing of no more than 10%.

Preferably, the acacia honey sample to be identified is detected by an evaporative light-scattering detector (ELSD) through high-performance liquid chromatography (HPLC).

Specifically, plotting a standard curve by a linear relationship between the turanose content and a peak area; according to the standard curve and an obtained peak area of the turanose detected in the acacia honey sample to be identified to calculate a turanose content.

In the present disclosure, the turanose content is a mass percentage relative to the acacia honey sample to be identified.

The identification method includes: detecting an acacia honey sample to be identified by an ELSD through HPLC; if there is a characteristic peak corresponding to turanose in an obtained spectrogram, with a content of no less than 0.5%, determining that the acacia honey sample to be identified is mature acacia honey.

Preferably, the detecting by the ELSD through the HPLC is conducted under liquid chromatography (LC) conditions as follows: using a Hi-Plex Pb, USP L19 chromatographic column at a column temperature of 68° C. to 72° C. (preferably 70° C.).

The Hi-Plex Pb, USP L19 chromatographic column is specifically an Agilent Hi-Plex Pb, USP L19, 4.0 mm×250 mm.

The LC refers to isocratic elution that is conducted using water as a mobile phase

When using the above LC conditions, the mobile phase has a flow rate of preferably 0.5 mL/min to 0.7 mL/min, more preferably 0.6 mL/min, and an injection volume of preferably 45 μL to 55 μL, more preferably 50 μL.

Preferably, in the detecting by the ELSD through the HPLC, the ELSD has the following parameters: an atomization temperature of 90° C., an evaporation temperature of 60° C., and a carrier gas flow rate of 1.2 standard litre per minute (SLM).

Preferably, before the detecting by the ELSD through the HPLC is conducted, a step of conducting a pretreatment on the acacia honey sample is preformed; where the pretreatment includes: dissolving the acacia honey sample in water, and conducting filtration.

Specifically, dissolving the acacia honey sample in ultrapure water, and mixing evenly to obtain a acacia honey sample solution; and the obtained acacia honey sample solution is directly subjected to ELSD detection through HPLC after being filtered through a filter membrane.

As an embodiment of the present disclosure, water with a volume 3 to 5 times a mass of the acacia honey sample is added to obtain an acacia honey sample solution. In an example, the pretreatment of the honey sample includes: taking 1 g of the acacia honey sample, adding with 3 mL of ultrapure water, conducting vortex, balancing to 5 mL, filtration with a 0.22 μm nylon filter membrane, and injection.

The beneficial effects of the present disclosure are as follows: in the present disclosure, it is found for the first time that the content change of turanose during the maturing of acacia honey has obvious characteristics. Moreover, it is determined that the content of turanose no less than 0.5% can be used as the standard to determine mature acacia honey. This indicator is used to distinguish mature acacia honey from immature acacia honey, and determine its maturity.

The present disclosure develops an identification method of maturity of acacia honey by using turanose as a characteristic standard. The method can detect turanose in acacia honey samples by an HPLC-based ELSD. The method has high specificity and sensitivity, and shows a limit of detection for turanose reaching 0.1%. Further, by optimizing the key parameters of the ELSD detection through HPLC, an accuracy of the detection of turanose is further enhanced in the acacia honey sample.

In the present disclosure, the identification method of maturity of acacia honey has simple operation, high efficiency, and low cost. The identification method also shows reliable and stable detection results and high degree of determination, and is convenient for popularization and application. The method provides an effective way for the identification of mature acacia honey, and has certain guiding significance for the formulation of related industries or national standards in high-quality acacia honey. In addition, the method is of great significance for protecting the legitimate rights and interests of honey consumers and maintaining the healthy development of the honey consumption industry.

### BRIEF DESCRIPTION OF THE DRAWINGS

FIG. 1 shows a chromatogram of acacia honey with different maturities in Example 1 of the present disclosure.

### DETAILED DESCRIPTION OF THE EMBODIMENTS

The following examples are intended to illustrate the present disclosure, but not to limit the scope of the present disclosure.

Unless otherwise specified, the experimental methods used in the following examples are conventional methods. The materials, reagents, and the like used in the examples are all commercially available, unless otherwise specified. Some of the instruments and reagents include: HPLC-based ELSD, Agilent Technologies, USA; electronic analytical balance (PL203), METTLERTOLEDO, Germany; ultrapure water machine (Milli-Q Gradient), Millipore, USA; vortex instrument (G560E), Scientific Industries, USE.

### Example 1 Content Detection of Turanose in Acacia Honey of Different Maturities

1. Source of samples: in 2019, 2020, and 2021, samples of acacia honey were collected from different bee farms in Yan'an, Shaanxi and Sanmenxia, Henan (the main production area of acacia honey), which were brewed by bees for 1 d, 3 d, 5 d, 7 d, 9 d, 11 d, and 13 d. For the samples of different brewing days, 3 samples were taken from each bee farm, and the brewing time and honeycomb sealing were recorded. All samples collected from 1 d to 5 d were manually concentrated to control their moisture content at 18% to 21%. The samples collected from 7 d to 13 d needed to measure moisture content, and the moisture content should be no more than 21%, and the honeycomb sealing needed to be no less than 75%.

The specific information of acacia honey samples was shown in Table 1 and Table 2. All acacia honey samples were tested for routine quality indicators and pollen identification, and the results showed that all samples were consistent with the attributes of the corresponding acacia honey.

All collected acacia honey samples were tested for turanose content.

2. Sample pretreatment: 1 g of the acacia honey sample was added with 3 mL of ultrapure water, vortexed, balanced to 5 mL, filtered with a 0.22 μm nylon filter membrane, and injected.

3. Instrument condition setting:

(1) LC conditions:
chromatographic column: Agilent Hi-Plex Pb, USP L19, 4.0 mm×250 mm;
column temperature: 70° C.;
injection volume: 50 μL;
mobile phase: ultrapure water;
elution conditions: isocratic elution; and
flow rate: 0.6 mL/min.

(2) ELSD conditions:
atomization temperature: 90° C.;
evaporation temperature: 60° C.;
carrier gas flow rate: 1.2 SLM; and
collection time period: no collection at 0-9 min (to avoid interference from high levels of fructose and glucose in honey), and data collection is conducted at 9-15 min.

4. Plotting of standard curve: standard substances of turanose with concentrations of 0.02%, 0.1%, 0.2%, 0.5%, 1.0%, 2.0%, and 5.0% were prepared, and the logarithm of a concentration value (10 was a base logarithm) and the logarithm of a response value were calculated to draw a standard curve.

5. Detection of turanose content in acacia honey samples: a standard curve was plotted by a linear relationship between the turanose content and a peak area; according to the standard curve and an obtained peak area of the turanose detected in the acacia honey sample, a turanose content was calculated.

According to statistics, the contents of turanose in acacia honey samples with different maturities were shown in Table 1 and Table 2. According to the above detection results, the content of turanose in the acacia honey sample was no less than 0.5%, and it was determined that the sample was mature acacia honey. This was consistent with the actual sampling results and met the conditions for mature acacia honey, that is, the acacia honey should be brewed in the hive for at least 7 d, and showed honeycomb sealing of no less than 75%. The content of turanose in the acacia honey sample was less than 0.5% and no less than 0.3%, and it was determined that the sample was semi-mature acacia honey. This sample was brewed in the hive for more than 3 d but no more than 5 d, and showed honeycomb sealing of 30% to 70%. The content of turanose in the acacia honey sample was less than 0.3%, and it was determined that the sample was immature acacia honey (the sample of 1 d to 3 d, basically showing no honeycomb sealing).

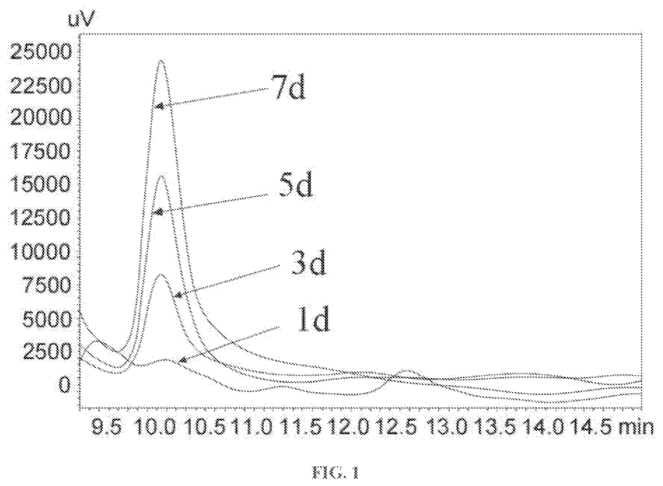

The detection spectrum of acacia honey with different maturities was shown in FIG. 1.

TABLE 1
Turanose content (%) and honeycomb sealing (%) in acacia honey samples of different years and different
days of mature in Yan'an, Shaanxi (where samples of 1 d to 13 d were randomly collected from bee farms
in each of 3 different regions, 3 parts for each sample, 63 samples per year, and 189 samples for 3 years)

| | 1 d | 3 d | 5 d | 7 d | 9 d | 11 d | 13 d |
|---|---|---|---|---|---|---|---|
| Year | n = 9 | n = 9 | n = 9 | n = 9 | n = 9 | n = 9 | n = 9 |
| 2019 | Turanose: ≤0.1% | Turanose: | Turanose: | Turanose: | Turanose: | Turanose: | Turanose: |
| | honeycomb | 0.15% ± 0.04% | 0.44% ± 0.04% | 0.56% ± 0.04% | 0.68% ± 0.06% | 0.83% ± 0.06% | 1.04% ± 0.1% |
| | sealing: | honeycomb | honeycomb | honeycomb | honeycomb | honeycomb | honeycomb |
| | None | sealing: | sealing: | sealing: | sealing: | sealing: | sealing: |
| | | 8% | 35% | 79% | 85% | 95% | 96% |
| 2020 | Turanose: | Turanose: | Turanose: | Turanose: | Turanose: | Turanose: | Turanose: |
| | 0.13% ± 0.02% | 0.16% ± 0.03% | 0.42% ± 0.03% | 0.63% ± 0.08% | 0.73% ± 0.07% | 0.94% ± 0.10% | 1.30% ± 0.23% |
| | honeycomb | honeycomb | honeycomb | honeycomb | honeycomb | honeycomb | honeycomb |
| | sealing: | sealing: | sealing: | sealing: | sealing: | sealing: | sealing: |
| | 3% | 9% | 44% | 83% | 86% | 93% | 96% |
| 2021 | Turanose: ≤0.1% | Turanose: | Turanose: | Turanose: | Turanose: | Turanose: | Turanose: |
| | honeycomb | 0.21% ± 0.03% | 0.37% ± 0.06% | 0.72% ± 0.10% | 0.81% ± 0.09% | 1.03% ± 0.14% | 1.21% ± 0.21% |
| | sealing: | honeycomb | honeycomb | honeycomb | honeycomb | honeycomb | honeycomb |
| | None | sealing: | sealing: | sealing: | sealing: | sealing: | sealing: |
| | | 10% | 39% | 78% | 88% | 95% | 95% |

TABLE 2
Turanose content (%) and honeycomb sealing (%) in acacia honey samples of different years and different days of
mature in Sanmenxia acacia production area, Henan (where samples of 1 d to 13 d were randomly collected from bee
farms in each of 3 different regions, 3 parts for each sample, 63 samples per year, and 189 samples for 3 years)

| | 1 d | 3 d | 5 d | 7 d | 9 d | 11 d | 13 d |
|---|---|---|---|---|---|---|---|
| Year | n = 9 | n = 9 | n = 9 | n = 9 | n = 9 | n = 9 | n = 9 |
| 2019 | Turanose: | Turanose: | Turanose: | Turanose: | Turanose: | Turanose: | Turanose: |
| | 0.16% ± 0.05% | 0.23% ± 0.04% | 0.39% ± 0.06% | 0.63% ± 0.06% | 0.73% ± 0.07% | 0.79% ± 0.06% | 0.99% ± 0.1% |
| | honeycomb | honeycomb | honeycomb | honeycomb | honeycomb | honeycomb | honeycomb |
| | sealing: | sealing: | sealing: | sealing: | sealing: | sealing: | sealing: |
| | 6% | 10% | 54% | 80% | 85% | 90% | 92% |
| 2020 | Turanose: | Turanose: | Turanose: | Turanose: | Turanose: | Turanose: | Turanose: |
| | 0.20% ± 0.07% | 0.25% ± 0.03% | 0.41% ± 0.05% | 0.70% ± 0.08% | 0.86% ± 0.08% | 0.94% ± 0.10% | 1.13% ± 0.15% |
| | honeycomb | honeycomb | honeycomb | honeycomb | honeycomb | honeycomb | honeycomb |
| | sealing: | sealing: | sealing: | sealing: | sealing: | sealing: | sealing: |
| | 7% | 10% | 65% | 77% | 88% | 92% | 93% |
| 2021 | Turanose: ≤0.1% | Turanose: | Turanose: | Turanose: | Turanose: | Turanose: | Turanose: |
| | honeycomb | 0.16% ± 0.03% | 0.36% ± 0.04% | 0.62% ± 0.04% | 0.77% ± 0.07% | 0.90% ± 0.10% | 0.96% ± 0.11% |
| | sealing: | honeycomb | honeycomb | honeycomb | honeycomb | honeycomb | honeycomb |
| | None | sealing: | sealing: | sealing: | sealing: | sealing: | sealing: |
| | | 9% | 68% | 79% | 80% | 82% | 83% |

### Example 2 Turanose as a Characteristic Marker in Identification of Commercially Available Acacia Honey

### 1. Source of Samples

A total of 18 honey samples of various grades or brands labeled as acacia honey were purchased from the market, and all samples were tested for pollen to prove that they were acacia honey.

2. Sample pretreatment: 1 g of the acacia honey sample to be identified was added with 3 mL of ultrapure water, vortexed, balanced to 5.0 mL, filtered with a 0.22 μm nylon filter membrane, and injected.

3. Instrument condition setting:

(1) LC conditions:
chromatographic column: Agilent Hi-Plex Pb, USP L19, 4.0 mm×250 mm;
column temperature: 70° C.;
injection volume: 50 μL;
mobile phase: ultrapure water;
elution conditions: isocratic elution; and
flow rate: 0.6 mL/min.

(2) ELSD conditions:
atomization temperature: 90° C.;
evaporation temperature: 60° C.;
carrier gas flow rate: 1.2 SLM.

4. Plotting of standard curve: standard substances of turanose with concentrations of 0.02%, 0.1%, 0.2%, 0.5%, 1.0%, 2.0%, and 5.0% were prepared, and the logarithm of a concentration value (10 was a base logarithm) and the logarithm of a response value were calculated to draw a standard curve.

5. Detection of turanose content in acacia honey samples: a standard curve was plotted by a linear relationship between the turanose content and a peak area; according to the standard curve and an obtained peak area of the turanose detected in the acacia honey sample, a turanose content was calculated.

The results were shown in Table 3.

TABLE 3
Determination of turanose content in
acacia honey sampled in the market

| Sample | Turanose content |
|---|---|
| No. | (%) |
| 1 | 0.45 |
| 2 | 0.16 |
| 3 | <0.1% |
| 4 | <0.1% |
| 5 | <0.1% |
| 6 | <0.1% |
| 7 | 0.55 |
| 8 | <0.1% |
| 9 | 0.63 |
| 10 | 0.35 |
| 11 | 0.43 |
| 12 | 0.87 |
| 13 | 0.64 |
| 14 | 0.36 |
| 15 | 0.73 |
| 16 | 1.21 |
| 17 | 0.23 |
| 18 | 0.76 |

The results showed that the 7 samples had a turanose content of greater than 0.5%, and were determined as mature acacia honey. According to the sample traceability information, it was confirmed that the samples came from certified enterprises producing mature acacia honey. This indicated that the identification method of maturity provided by the present disclosure showed a high discrimination accuracy. For other samples, 4 samples had a turanose content between 0.3 to 0.5%, and were determined as semi-mature acacia honey; 7 samples had a turanose content of less than 0.3%, and were determined as immature acacia honey. The above results proved that the identification method of the present disclosure could effectively identify the maturity of acacia honey. Therefore, the method can be used for grading the maturity of commercial acacia honey, so as to realize different pricing of acacia honey according to the maturity.

Although the present disclosure has been described in detail above with general description and specific embodiments, some modifications or improvements can be made on the basis of the present disclosure, which will be apparent to those skilled in the art. Therefore, all of these modifications or improvements made without departing from the spirit of the present disclosure fall within the claimed scope of the present disclosure.

What is claimed is:

1. (canceled)

2. A method for identifying maturity of acacia honey, comprising using turanose as a characteristic marker of mature acacia honey: wherein if the acacia honey sample to be identified has a turanose content of no less than 0.5%, it is determined that the acacia honey sample to be identified is mature acacia honey: if the acacia honey sample to be identified has a turanose content of no less than 0.3% but less than 0.5%, it is determined that the acacia honey sample to be identified is semi-immature acacia honey; and if the acacia honey sample to be identified has a turanose content of less than 0.3%, it is determined that the acacia honey sample to be identified is immature acacia honey.

3. A method for identifying mature acacia honey and immature acacia honey, comprising using turanose as a characteristic marker of mature acacia honey: wherein if the acacia honey sample to be identified has a turanose content of no less than 0.5%, it is determined that the acacia honey sample to be identified is mature acacia honey: if the acacia honey sample to be identified has a turanose content of no less than 0.3% but less than 0.5%, it is determined that the acacia honey sample to be identified is semi-immature acacia honey; and if the acacia honey sample to be identified has a turanose content of less than 0.3%, it is determined that the acacia honey sample to be identified is immature acacia honey.

4. An identification method of maturity of acacia honey, comprising the following steps: detecting an acacia honey sample using turanose as a characteristic marker; wherein if the acacia honey sample to be identified has a turanose content of no less than 0.5%, it is determined that the acacia honey sample to be identified is mature acacia honey; if the acacia honey sample to be identified has a turanose content of no less than 0.3% but less than 0.5%, it is determined that the acacia honey sample to be identified is semi-immature acacia honey; and if the acacia honey sample to be identified has a turanose content of less than 0.3%, it is determined that the acacia honey sample to be identified is immature acacia honey;
the mature acacia honey is brewed in a hive for at least 7 d, and shows honeycomb sealing of no less than 75%;
the semi-immature acacia honey is brewed in the hive for more than 3 d but no less than 5 d, and shows honeycomb sealing of 30% to 70%; and
the immature acacia honey is brewed in the hive for 0 d to 3 d, and shows honeycomb sealing of no more than 10%.

5. The identification method of maturity of acacia honey according to claim 4, wherein the acacia honey sample is detected by an evaporative light-scattering detector (ELSD) through high-performance liquid chromatography (HPLC).

6. The identification method of maturity of acacia honey according to claim 5, wherein a process of detecting the acacia honey sample comprises: detecting the acacia honey sample by the ELSD through the HPLC; plotting a standard curve by a linear relationship between the turanose content and a peak area; according to the standard curve and an obtained peak area of the turanose detected in the acacia honey sample, calculating an turanose content; wherein the turanose content is a mass percentage relative to the acacia honey sample.

7. The identification method of maturity of acacia honey according to claim 5, wherein the detecting by the ELSD through the HPLC is conducted under liquid chromatography (LC) conditions as follows: using a Hi-Plex Pb, USP L19 chromatographic column at a column temperature of 68° C. to 72° C.

8. The identification method of maturity of acacia honey according to claim 7, wherein the Hi-Plex Pb, USP L19 chromatographic column is a 4.0 mm×250 mm column.

9. The identification method of maturity of acacia honey according to claim 7, wherein isocratic elution is conducted using water as a mobile phase.

10. The identification method of maturity of acacia honey according to claim 9, wherein the mobile phase has a flow rate of 0.5 mL/min to 0.7 mL/min and an injection volume of 45 μL to 55 μL.

11. The identification method of maturity of acacia honey according claim 5, wherein in the detecting by the ELSD through the HPLC, the ELSD has the following parameters: an atomization temperature of 90° C., an evaporation temperature of 60° C., and a carrier gas flow rate of 1.2 standard litre per minute (SLM).

12. The identification method of maturity of acacia honey according claim 5, further comprising a step of pretreatment on the acacia honey sample before the detecting by the ELSD through the HPLC is conducted; wherein the pretreatment comprises: dissolving the acacia honey sample in water, and conducting filtration.

13. The identification method of maturity of acacia honey according to claim 12, wherein water with a volume 3 to 5 times a mass of the acacia honey sample is added to obtain an acacia honey sample solution.

14. The identification method of maturity of acacia honey according to claim 7, wherein a process of detecting the acacia honey sample comprises: detecting the acacia honey sample by the ELSD through the HPLC; plotting a standard curve by a linear relationship between the turanose content and a peak area; according to the standard curve and an obtained peak area of the turanose detected in the acacia honey sample, calculating an turanose content; wherein the turanose content is a mass percentage relative to the acacia honey sample.

15. The identification method of maturity of acacia honey according to claim 11, wherein a process of detecting the acacia honey sample comprises: detecting the acacia honey sample by the ELSD through the HPLC; plotting a standard curve by a linear relationship between the turanose content and a peak area; according to the standard curve and an obtained peak area of the turanose detected in the acacia honey sample, calculating an turanose content; wherein the turanose content is a mass percentage relative to the acacia honey sample.

16. The identification method of maturity of acacia honey according to claim 12, wherein a process of detecting the acacia honey sample comprises: detecting the acacia honey sample by the ELSD through the HPLC; plotting a standard curve by a linear relationship between the turanose content and a peak area; according to the standard curve and an obtained peak area of the turanose detected in the acacia honey sample, calculating an turanose content; wherein the turanose content is a mass percentage relative to the acacia honey sample.
